(19) United States
(12) Patent Application Publication
(10) Pub. No.: US 2006/0246163 A1
(43) Pub. Date: Nov. 2, 2006

(54) TREATMENT OF INSULIN RESISTANCE SYNDROME

(75) Inventors: Tae-Lin Huh, Daegu (KR); Hebok Song, Seoul (KR); Dong-Chan Park, Daegu (KR); Seung Lark Hwang, Daegu (KR)

Correspondence Address: JHK LAW, P.O. BOX 1078, LA CANADA, CA, 91012-1078, US

(73) Assignee: TG BIOTECH, Daegu (KR)

(21) Appl. No.: 11/380,472

(22) Filed: Apr. 27, 2006

Related U.S. Application Data
(60) Provisional application No. 60/675,703, filed on Apr. 27, 2005.

(51) Int. Cl. A61K 36/10

(52) U.S. Cl. 424/762

(57) ABSTRACT

The present application describes a composition that includes an extract of Gynostemma pentaphyllum used to treat insulin resistance syndrome, obesity, hypertriglyceridemia, as well as decrease body fat mass.

### CROSS-REFERENCE To RELATED APPLICATIONS

The present application claims benefit of priority to U.S. Provisional Application No. 60/675,703, filed Apr. 27, 2005, the contents of which are incorporated by reference herein in its entirety.

### BACKGROUND OF THE INVENTION

1. Field of the Invention:

The present invention relates to a therapeutic composition comprising gypenosides or the extracts of Gynostemma pentaphyllum (G. pentaphyllum). The present invention further relates to the use of such a therapeutic composition for treating the symptoms of insulin resistance syndrome, obesity and hypertriglyceridemia.

2. General Background and State of the Art:

Insulin resistance syndrome is a complex and polygenic disease. The two important factors, obesity and inflammation, have been implicated in the development of the syndrome and related conditions. Visceral obesity, especially, is closely related to the development of insulin resistance. Considering a wide spread patients of insulin resistance syndrome either with obvious clinical symptoms or not and its serious end results when untreated, there exists a medicinal need for an effective and safe oral medication to treat insulin resistance syndrome.

Years of both basic and clinical researches to identify mechanisms underlying insulin resistance in muscle tissues derived from obesity uncovered that fatty acid accumulation inside cells is the major culprit for the development of the syndrome (McGarry, 2002). Once circulating fatty acids (FA) enter cell through fatty acid transport protein (FATP), FA become esterified with coenzyme A to form acyl-CoA. Subsequently, acyl-CoA (partly conjugated to glycerol backbone to make diacylglycerol) activates c-Jun N-terminal kinase (JNK) and/or protein kinases Cs (PKCs). These kinases then directly or indirectly phosphorylated serine residues of insulin receptor substrate 1 and 2 (IRS1/2) (Dresner, 1999). Employing I-kappa B kinase (IKK)-β knock-out mice and using salicylate, an inhibitor of IKKβ, Schulman and his colleagues discovered that serine phosphorylation on IRS1 induced by lipid in muscle tissue of insulin resistance syndrome patients is mediated by IKKβ activity (Kim, 2001). The serine phosphorylated IRS1/2 hinders recruitment of phosphatidyl-inositol 3 kinase (PI3K) to insulin receptor. Disengaged PI3K does not transfer insulin signal to glucose transporter, especially insulin-sensitive glucose transporter 4 (GLUT4), and consequently insulin resistance develops in muscle tissues. Recently, obesity-related insulin resistance in other important insulin target tissues, such as liver and adipose tissues, has been postulated to have a surprisingly different mechanism (Ozcan, 2004). The combined picture of obesity-derived insulin resistance in either skeletal muscle or hepatocyte/adipocyte, however, ultimately converges to insulin insensitive GLUT4, which functions to remove sugar units from circulation.

Glucose is a preferable energy source in most tissues to produce ATP. The carbohydrate is hydrophylic and cannot enter cells freely. The gate of glucose is glucose transporter (GLUT). More than a dozen diverse GLUTs have been discovered in mammalian tissues. Some of them are localized on specific tissues, while some are widely distributed in numerous types of tissues. Skeletal muscles, liver and adipose tissues are the major glucose disposing organs after a meal. In other words, the three organs are insulin sensitive. When large quantity of carbohydrate-rich food is consumed in excess of immediate requirements, acutely up-risen glucose level needs to be stored as glycogen or fat (triacylglycerol) for later uses. Skeletal muscles and hepatocytes are where surplus glucose is stored as glycogen. Liver and fat cells (adipocytes) are both major organs that synthesize and store fat. During exercise, periods of stress or starvation, triacylglycerol in adipose tissue is hydrolyzed to free fatty acids by lipolysis for oxidation as a respiratory fuel. The free fatty acids transported to muscle and liver tissues are further oxidized by β-oxidation to generate NADH, which is required for ATP synthesis.

Reducing body fat mass is beneficial for improvement of insulin resistance and type 2 diabetes since obesity frequently accompanies type 2 diabetes and higher levels of blood free fatty acids, which reduce insulin signaling. Therefore, there are many concerns regarding how to and which part of fat mass to reduce in body. Activation of AMP-activated protein kinase (AMPK) is critical to improve insulin resistance since it can reduce body fat synthesis but increases β-oxidation: activation of AMPK results in inactivation of acetyl-CoA carboxylase (ACC), by which, in turn, malonyl-CoA production is reduced, leading to decrease of fat synthesis but increase of β-oxidation (Oh, 2005). For β-oxidation, fatty acid should be transported into mitochondria. A transport system, the carnitine shuttle, is needed to enable long-chain fatty acid to cross the mitochondrial membranes. In the liver and muscle, this transport system is inhibited by malonyl-CoA. Therefore, decreased level of malonyl-CoA caused by activation of AMPK stimulates transport of fatty acid into mitochondria, increases β-oxidation, and decreases body fat mass.

Glucose availability signals pancreatic β-cells to secrete insulin. Insulin stimulates translocation of GLUT4 to plasma membranes to promote glucose uptake into cells, and also increases glycogen synthesis. In case insulin does not properly activate organs to dispose of the circulating glucose, pancreatic β-cells secrete more insulin to adjust glucose level within the physiological range. The overloaded β cells and the insensitiveness to insulin of the glucose disposing organs are important features of insulin resistance. The continued stress becomes eventually type 2 diabetes. Unfortunately in the middle of developing the disease, hypertension, atherosclerosis and other disorders are accompanied with type 2 diabetes.

Among currently prescribed drugs for type 2 diabetes, metformin, a biguanide, and rosiglitazone, a thiazolidinedione (TZD), improve insulin sensitivity. Metformin has been used clinically for decades and its anti-diabetic mechanism depends on its inhibitory activity of gluconeogenesis in the liver. TZDs are known to be ligands of peroxisome proliferator-activated receptor (PPAR)-γ, which recognizes a broad spectrum of fatty acids and their derivatives. Upon binding to PPAR γ, TZDs modulate a variety of genes related to adipogenesis. Fatty acids and peptide hormones derived from adipose tissue are known to mediate the TZD-induced improvement of insulin sensitivity. Interestingly, despite differences in their origin and mode of action, the two compounds share a common ground of stimulating AMPK activity by elevating AMP versus ATP level by inhibiting enzyme activity of respiratory complex 1 of mitochondrial respiratory chain (Brunmair 2004). However, whether the stimulated AMPK activity by these two hypoglycemic compounds contributes to the improvement of insulin sensitivity is not known. Nonetheless, there is a common sense that elevated AMPK activity improves hyperglycemic condition.

An idea that compounds activating AMPK activity can be candidates for the treatment of obesity, insulin resistance syndrome or type 2 diabetes prompted us to develop an inventive screening method. Plant materials, which have been known to be safely used for hundreds years in Asia for the treatment of various diseases, were examined to determine whether they activate AMPK in muscle cells. Among thousands of plant extracts that were tested, the extract of G. pentaphyllum was selected as a plausible candidate for treating insulin resistance syndrome.

G. pentaphyllum, a perennial herb belonging to the family Cucurbitaceae, has been used as a folk medicine since this plant extract is believed to contain chemicals or ingredients that may lower cholesterol level, regulate blood pressure, stimulate immune system, reduce inflammation, hinder the stickiness of platelets and so forth. However, all of these potential effects remain to be scientifically elucidated or proved. G. pentaphyllum is also called Amachzuru, Jiaogulan, Miracle Grass, Southern Ginseng, Vitis pentaphyllum, and Xianxao. The primary constituents of extracts of these leaves are gypenosides (GP), which are dammarane-type saponins. Recently, Liu and colleagues (Liu, 2004) isolated 15 dammarane-type saponins from G. pentaphyllum. Ten were already isolated previously and five of them were new triterpenoids bearing a side-chain at C-17 with an epoxy ring. From the same plant, Yin and others (Yin, 2004) also reported that they have found 15 new dammarane-type glycosides among 19 isolates. These new recent successes in new compound discoveries can be attributed to the development of modern analytical tools. However, whether each or combinations of newly isolated glycosides have any specific pharmacological activity or not has not been elucidated.

Extracts of G. pentaphyllum showed good effects against insulin resistance syndrome. The anti-insulin resistance activity of the extract or GP are based on two important discoveries. Firstly, the extract stimulated AMPK activity, which is a well-known stimulator for glucose transporter 4 (GLUT4) translocation to the plasma membrane in an insulin-independent manner. Secondly, the extract suppressed IKK (inhibitor of I-κB kinase)-β and JNK (c-Jun N-terminal kinase) activities, resulting in reduction of serine phosphorylation of IRS1.

### SUMMARY OF THE INVENTION

This invention relates to the usage of an herbal extract containing dammarane-type saponins, named gypenosides (GP), from G. pentaphyllum.

Another aspect of this invention is a process for preparing the herbal extract. This method comprises extracting herbal component, and drying the extract eluates.

The present invention provides a method of using G. pentaphyllum extract or gypenosides for lowering blood glucose level after a meal in subjects having insulin resistance syndrome by stimulating glucose uptake into cells by stimulating GLUT4 translocation to plasma membrane in an insulin-independent manner and reducing insulin resistance by repressing IKKβ and JNK activities.

The invention also provides for use of G. pentaphyllum extract or GP for increasing disposal of body fat/lipid by stimulating AMPK activity and subsequently inactivating ACC (acetyl CoA carboxylase) activity, resulting in an increase of β-oxidation.

The invention also provides for use of G. pentaphyllum extract or GP for increasing insulin signaling by inhibiting IKKβ and JNK activities in muscle tissue. Inhibition of these kinase activities reduces phosphorylation of serine residues in IRS, thus increasing insulin-stimulated glucose uptake into cells.

The invention also provides for methods for preventing or treating insulin resistance and related disorders comprising administering G. pentaphyllum extract or GP to a subject in need thereof suffering from the effects of insulin resistance syndrome.

Thus, in one aspect, the invention is directed to a composition comprising an insulin resistance syndrome, obesity, decreasing body fat mass and hypertriglyceridemia treating effective amount of an extract of Gynostemma pentaphyllum. The composition may include gypenosides in a concentration of about 0.5 to 10% by weight. Further, the amount of gypenosides in the composition may be about 10 to 2,000 μg/ml μg/ml.

The invention is also directed to a method for treating symptoms of insulin resistance syndrome obesity/overweight and hypertriglyceridemia in a subject administering to the subject a therapeutically effective amount of the above composition. The amount of the extract used may be 10 mg to 30 g per day or about 0.5 g to 5 g per day.

In another aspect, the invention is directed to a method for treating symptoms of insulin resistance syndrome, obesity/overweight, decreasing body fat mass and hypertriglyceridemia in a subject comprising administering to the subject a therapeutically effective amount of the composition described above. The amount of the composition may be about 1 to 1000 mg per day or 10 to 800 mg per day.

The above composition may include an aqueous carrier such as spring water, filtered water, distilled water, carbonated water, juice, yogurt, milk, edible oils and a combination thereof. The composition may be included as food additives, such as ice cream, hamburger, cereals, cookies, breads, cakes, biscuits, meat product, or a combination thereof. In addition, the composition may include a preservative agent, sweetener, flavoring agent, coloring agent, or a combination thereof Further, the composition may be formulated into a tablet. And the tablet may be made from a base selected from a filler, binder, coating, excipients, or a combination thereof The base may further include plant cellulose, natural silica, magnesium sterate, wax, vegetable glycerides, vegetable stearate or a combination thereof The composition may also include a compound of glitazones, fibrates, statins, biguanides, sulfonylureas, adenine nucleotides, or their derivatives, and pharmaceutically acceptable salts thereof.

In another aspect, the invention is also directed to a method for selecting non-toxic AMPK activators, which have adipogenesis enhancing activity in 3T3-L1 cells.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows that GP treatment increases adipocity in 3T3-L1 cells in the presence of hormones. 1) no treatment; 2) 5 μg/mL; 3) 20 μg/mL; 4) 50 μg/mL of GP.

FIG. 2 shows additive effect of GP with rosiglitazone in enhancing adipogenesis in 3T3-L1 cells.

FIG. 3 shows up-regulation of PPARγ and GLUT4 in cytosol and membrane fraction, respectively, by G. pentaphyllum extract in rat vascular smooth muscle cells. 1) control; 2) rosiglitazone, 5 μM; 3) G. pentaphyllum extract, 0.5 mg/mL

FIGS. 4A-4I show that GP triggers GLUT4 translocation to plasma membrane in L6 myotube cells. L6 cells were induced to mature myotube cells under low glucose media for 9 days. The cells then treated with either GP (60 μg/mL) or insulin (100 nM) with or without an inhibitor of either phosphatidylinositol 3 kinase (PI3K) or p38 MAPK. The inhibitors were added 1 h before treating GP or insulin. After fixed and washed in cold PBS extensively, the cells were incubated with specific GLUT4 antibodies and followed by an incubation of FITC-conjugated secondary antibodies. The cells were then analyzed in FACS machine. A) whole cell distribution profile in FACS (The circle in figure A denotes gated area.); B) control cells with no treatment; C) cells treated with GP and incubated with nonspecific antibodies; D) cells treated with gypenosides and reacted with GLUT4 specific antibodies; E) cells treated with GP in the presence of wortmanin; F) cells treated with GP in the presence of SB20358; G) cells triggered with insulin H) cells with insulin in the presence of wortmanin I) cells with insulin in the presence of SB20358.

FIG. 5 shows time dependent activation of AMPK by GP. L6 myotube cells were treated with 60 μg/mL of GP and incubated for the period of time indicated. Cells were lysed in lysis buffer and cytosolic proteins were resolved by SDS-PAGE, protein bands were transferred onto a nitrocellulose membrane, and phospho-AMPK was analyzed with specific antibodies. 1) control cells with no treatment; 2) cells treated with GP for 30 min; 3) cells treated with GP for 1 h; 3) cells with treated with GP for 2 hs.

FIG. 6 shows that AMPK phosphorylation was induced by GP in the presence of high glucose in rat vascular smooth muscle cells. 1) control; 2) high glucose (27.5 mM); 3) high glucose +GP 10 μg/mL; 4) high glucose+GP 30 μg/mL.

FIG. 7 shows effect of GP on AMPK and p38 MAPK activities in L6 muscle cells in the presence of high glucose. The kinase activities were evaluated with specific antibodies. 1) control; 2) GP 30 μg/mL; 3) GP 60 μg/mL; 4) AICAR 1 mM.

FIG. 8 shows effect of GP on ACC and AKT phosphorylation in L6 cells. 1) control; 2) GP 30 μg/mL; 3) GP 60 μg/mL; 4) GP 100 μg/mL; 5) AICAR 1 mM; 6) insulin 100 nM.

FIG. 9 shows effect of GP on the serine phosphorylation of IRS1 in L6 cells. Cells were pretreated with fatty acid-conjugated BSA to induce insulin resistant state in vitro. 1) BSA; 2) BSA+fatty acids; 3) BSA+fatty acids+GP 60 μg/mL.

FIG. 10 shows effect of GP on the serine phosphorylation of IRS1, IKKβ, and SAPK/JNK in L6 myotube cells in the presence of tunicamycin. Tunicamycin, an antibiotic known to inhibit N-linked glycosylation, forces cells into an insulin-resistant state. The cytosolic fraction was subjected on SDS-PAGE and blotted on nitrocellulose membrane. The membrane was incubated with anti-phospho-IKKβ (S^{177/181}), anti-phospho IRS1 (S³⁰⁷), and anti-phospho SAPK/JNK (T¹⁸³) antibodies. 1) control cells with no treatment; 2) L6 cells treated with tunicamycin; 3) cells treated with 30 μg/mL GP in the presence of tunicamycin; 4) cells treated with 60 μg/mL GP in the presence of tunicamycin; 5) cells treated with 1 mM AICAR in the presence of tunicamycin; 6) cells incubated with 100 nM insulin in the presence of tunicamycin; 7) cells incubated with 100 nM insulin in the absence of tunicamycin.

FIG. 11 shows GP reduced IKK activity and suppressed NF-κB activation in rat smooth muscle cells in the presence of high glucose. Upper panels: phospho I-κB (lanes 1-4) and p65 (lanes 5-7) Lower panels: beta tubulin (lanes 1-4) and nuclear actin (lanes 5-7) 1) control; 2) high glucose; 3) GP 10 μg/mL; 4) GP 30 μg/mL; 5) control; 6) GP 10 μg/mL; 7) GP 30 μg/mL.

FIG. 12 shows effect of GP on the JNK activity in L6 myotube cells. L6 myotube cells were treated with GP for 2 hs. Cytosolic fraction was immunoblotted against phospho JNK antibodies. 1) control cells with no treatment; 2) GP 30 μg/ mL; 3) GP 60 μg/ mL; 4) AICAR 1 mM; 5) insulin 100 nM.

FIG. 13 shows GP increased 2-deoxyglucose uptake in L6 muscle cells. 1) no treatment; 2) GP 60 μg/mL; 3) AICAR 1 mM; 4) insulin 100 nM.

FIG. 14 shows GP increased β-oxidation in HepG2 cells.

FIG. 15 shows improved glucose tolerance of db/db mice fed with GP.

FIG. 16 shows improved glycated hemoglobin level in mice fed with GP. abValues not sharing a common letter are significantly different among groups at p<0.05 HbA1c: Glycated hemoglobin.

FIG. 17 shows marked improvement in hyperinsulinemia of db/db mice orally fed with GP for 8 weeks. ^{abc}Values not sharing a common letter are significantly different among groups at p<0.05.

FIG. 18 shows C-peptide lowering effect of GP. ^{ab}Values not sharing a common letter are significantly different among groups at p<0.05.

FIG. 19 shows that administering GP to db/db mice reduced their leptin levels. ^{abc}Values not sharing a common letter are significantly different among groups at p<0.05.

FIG. 20 shows effect of GP on the hepatic phosphoenolpyruvate carboxykinase (PEPCK). ^{ab}Values not sharing a common letter are significantly different among groups at p<0.05.

FIG. 21 shows effect of GP on the AMPK activity and serine phosphorylation of insulin receptor substrate 1 of the skeletal muscle tissue. 1) control; 2) mouse fed with GP 0.01%; 3) mouse fed with GP 0.02%; 4) mouse fed with 0.02% Glucovance.

### DETAILED DESCRIPTION OF THE PREFERRED EMBODIMENTS

In the present application, “a” and “an” are used to refer to both single and a plurality of objects.

As used herein, “carriers” include pharmaceutically acceptable carriers, excipients, or stabilizers, which are nontoxic to the cell or mammal being exposed thereto at the dosages and concentrations employed. Often the pharmaceutically acceptable carrier is an aqueous pH buffered solution. Examples of pharmaceutically acceptable carriers include without limitation buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

As used herein, a “dose” refers to a specified quantity of a therapeutic agent prescribed to be taken at one time or at stated intervals.

As used herein, “effective amount” is an amount sufficient to effect beneficial or desired clinical or biochemical results. An effective amount can be administered one or more times. For purposes of this invention, an effective amount of a compound is an amount that is sufficient to palliate, ameliorate, stabilize, reverse, slow or delay the progression of the disease state. In a preferred embodiment of the invention, the “effective amount” is defined as an amount of compound capable of stimulating AMPK, and GLUT4 translocation. In yet another embodiment, the “effective amount” is defined as the amount of the composition that is effective to treat, treat the symptoms, cure or protect against obesity or insulin resistance syndrome. In other embodiments, “effective amount” may be that amount of gypenosides that increases glucose transport into the cell independent of insulin, where the effect of insulin resistance syndrome is sought to be lessened.

As used herein, “GP” refers to gypenosides extracted from G. pentaphyllum.

As used herein, “Insulin Resistance Syndrome” refers to various abnormalities associated with insulin resistance/compensatory hyperinsulinemia, which include the following: some degree of glucose intolerance (impaired fasting glucose and impaired glucose tolerance); dyslipidemia (increased triglycerides, decreased high-density lipoprotein cholesterol (HDL-C), decreased low-density lipoprotein (LDL)-particle diameter (small, dense LDL particles), and increased postprandial accumulation of triglyceride-rich lipoproteins); endothelial dysfunction (increased mononuclear cell adhesion, increased plasma concentration of cellular adhesion molecules, increased plasma concentration of asymmetric dimethylarginine, and decreased endothelial-dependent vasodilatation); procoagulant factors (increased plaminogen activator inhibitor-1 and increased fibrinogen); hemodynamic changes (sympathetic nervous system activity and renal sodium retention); markers of inflammation (increased C-reactive protein, white blood cell count, etc.); abnormal uric acid metabolism (increased plasma uric acid concentration and renal uric acid clearance); increased testosterone secretion (ovary); and sleep-disordered breathing. Further, some of the clinical syndromes associated with insulin resistance include the following: type 2 diabetes, cardiovascular disease, essential hypertension, polycystic ovary syndrome, nonalcoholic fatty liver disease, certain forms of cancer, and sleep apnea.

As used herein “pharmaceutically acceptable carrier and/or diluent” includes any and all solvents, dispersion media, coatings antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” refers to both therapeutic treatment and prophylactic or preventative measures. Those in need of treatment include those already with the disorder as well as those in which the disorder is to be prevented. “Palliating” a disease means that the extent and/or undesirable clinical manifestations of a disease state are lessened and/or the time course of the progression is slowed or lengthened, as compared to a situation without treatment.

In one embodiment, the prepared G. pentaphyllum extract powder or GP provide previously unknown therapeutic or health promoting benefits. More particularly, the extract or GP, in a pharmacologically effective amount and regimen, can improve impaired glucose tolerance, impaired insulin resistance and impaired leptin resistance.

In a further embodiment, GP is used to activate AMPK. In another embodiment, the target protein is ACC. In a still further embodiment the target protein is intracellular protein carnitine palmitoyl transferase (CPT). In a further embodiment the target protein is membranous protein IRS 1. In a further embodiment the target protein is intracellular protein GLUT4.

Extract Compositions and Formulations

The amount of gypenoside in the inventive treatment composition may be in a concentration of about 0.5 to 10% by weight, or 0.6 to 9%, 0.7 to 8%, 0.8 to 7%, 0.9 to 6%, 1 to 5%, 2 to 4%, or more preferably 2.1 to 3.5%, 2.2 to 3.4%, 2.3 to 3.3%, 2.4 to 3.2%, 2.5 to 3%, 2.6 to 2.9%, or 2.7 to 2.8%.

The amount of gypenosides in the inventive treatment composition may be in a concentration of about 10 to 2,000 μg/ml, 20 to 1,000 μg/ml, 30 to 500 μg/ml, or more preferably 100 to 300 μg/ml.

In an alternate embodiment, an active composition may be made from a mixture of chromium, manganese, zinc, niacin, vitamin B6 and vitamin B12. Preferably, the chromium is present in an amount of about 20 to about 500 micrograms, manganese is present in an amount of about 1 to about 10 milligrams, zinc is present in an amount of about 2 to about 10 milligrams, niacin is present in an amount of about 50 to about 500 milligrams, vitamin B6 is present in an amount of about 1 to about 50 milligrams, and vitamin B12 is present in an amount of about 5 to about 100 micrograms per dose.

Depending on the specific clinical status of the disease, administration can be made via any accepted systemic delivery system, for example, via oral route or parenteral route such as intravenous, intramuscular, subcutaneous or percutaneous route, or vaginal, ocular or nasal route, in solid, semi-solid or liquid dosage forms, such as for example, tablets, suppositories, pills, capsules, powders, solutions, suspensions, cream, gel, implant, patch, pessary, aerosols, collyrium, emulsions or the like, preferably in unit dosage forms suitable for easy administration of fixed dosages. The pharmaceutical compositions will include a conventional carrier or vehicle and, in addition, may include other medicinal agents, pharmaceutical agents, carriers, adjuvants, and so on. In the invention, the carrier for the herbal composition may preferably include, a base of berries or fruit, a base of vegetable soup or bouillon, a soya-milk drink, or a nutritive supplement.

If a vegetable soup or bouillon base is desired to be used as a base for the herbal composition, it can be readily seen that any vegetable soup or bouillon base can be used, so long as the anti-diabetic effect of the herbal composition is maintained.

If it is desired that the base be made from extracts of berries or fruits, then it is understood that any berry or fruit base may be used so long as its use does not interfere with the anti-diabetic effectiveness of the herbal medicinal composition.

If the inventive composition is desired to be placed into soya milk, it is understood that such a drink will need to be refrigerated to prevent toxic effects. It is further understood that the inventive composition may be placed, mixed, added to or combined with any other nutritional supplement so long as the anti-insulin resistance effect of the herbal composition is maintained.

If desired, the pharmaceutical composition to be administered may also contain minor amounts of non-toxic auxiliary substances such as wetting or emulsifying agents, pH buffering agents and the like, such as for example, sodium acetate, sorbitan monolaurate, triethanolamine oleate, and so on.

The amount of the herbal medicine in a formulation can vary within the full range employed by those skilled in the art, e.g., from about 0.01 weight percent (wt %) to about 99.99 wt % of the medicine based on the total formulation and about 0.01 wt % to 99.99 wt % excipient.

The preferred mode of administration, for the conditions mentioned above, is oral administration using a convenient daily dosage regimen, which can be adjusted according to the degree of the complaint. For said oral administration, a pharmaceutically acceptable, non-toxic composition is formed by the incorporation of the herbal composition in any of the currently used excipients, such as, for example, pharmaceutical grades of mannitol, lactose, starch, magnesium stearate, sodium saccharine, talc, cellulose, glucose, gelatin, sucrose, magnesium carbonate, and the like. Such compositions take the form of solutions, suspensions, tablets, pills, capsules, powders, sustained release formulations and the like. Such compositions may contain between 0.01 wt % and 99.99 wt % of the active compound according to this invention.

In one embodiment, the compositions will have the form of a sugar coated pill or tablet and thus they will contain, along with the active ingredient, a diluent such as lactose, sucrose, dicalcium phosphate, and the like; a disintegrant such as starch or derivatives thereof; a lubricant such as magnesium stearate and the like; and a binder such as starch, polyvinylpyrrolidone, acacia gum, gelatin, cellulose and derivatives thereof, and the like.

It is understood that by “pharmaceutical composition” or “herbal medicinal composition”, it is meant that the herbal composition is formulated into a substance that is to be administered purposefully for treating or preventing insulin resistance syndrome, obesity and hypertriglyceridemia in an individual. The mode of action is believed to be by the activation of AMPK and reduction of IKK and JNK activities. However, it is understood that GP per se do not have a toxic effect.

Therapeutic Composition

The formulation of therapeutic compounds is generally known in the art and reference can conveniently be made to Remington's Pharmaceutical Sciences, 17th ed., Mack Publishing Co., Easton, Pa., USA. For example, from about 0.05 μg to about 20 mg per kilogram of body weight per day may be administered. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intra nasal, or intradermal.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, chlorobutanol, phenol, sorbic acid, themerosal and the like. In many cases, it will be preferable to include isotonic agents, for example, sugars or sodium chloride. Prolonged absorption of the injectable compositions can be brought about by the use in the composition of agents delaying absorption, for example, aluminium monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterile active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique, which yield a powder of the active ingredient plus any additional desired ingredient from a previously sterile-filtered solution thereof.

The active compound may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 μg/ml of carrier. In the case of compositions containing supplementary active ingredients, the dosages are determined by reference to the usual dose and manner of administration of the said ingredients.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Process of Preparing GP

In this embodiment is illustrated a process for preparing the herbal extract of the present invention. In the process of preparation, extracts from Gynostemma pentaphyllum with particular types of anti-hyperglycemic activity were selected. Extracts that contain high concentrations of pharmacologically active compounds, which comprise active ingredients in the herbal extract-based composition of the present invention were obtained.

In one embodiment, this method comprises the steps of:

(a) extracting macerated G. pentaphyllum leaves in aqueous alcohol (such as 70% ethanol);

(b) repeating step (a), recovering a second aqueous alcohol extract eluate, and pooling the two extracts;

(c) evaporating the alcohol and further mixing the solution with distilled water and filtering;

(d) further mixing the solution with 1-BuOH, water layer, and evaporating off 1-BuOH;

(e) recovering the organic material by reducing the liquid portion of pooled eluates by drying (e.g., air drying), and forming G. pentaphyllum extract powder; and

(f) isolating and refining further as needed.

### Example 2

### Identification of Compounds that Increase Adipogenecity in 3T3-L1 cells in vitro

Active compounds for adipogenecity enhancing activity were identified in a cell-based assay. Briefly, 3T3-L1 cells were cultured in 96 well plates. Prior to screening, cells were adjusted to optimal conditions to mature into adipocytes. Preadipocytes, 3T3-L1, maintained in DMEM containing 10% FCS were induced to mature adipocytes in the presence of a specified hormone cocktail (5 μg/mL insulin, 1 μM dexamethasone, and 500 μg/mL IBMX). At day 3, the medium containing only insulin as a hormone in DMEM was changed every other day. Routinely, the level of adipogenesis induction was estimated by staining in Oil-Red O. The concentration of each material tested was 10 μg/mL. Rosiglitazone was used as a positive control increasing adipogenesis in 3T3-L1 cells.

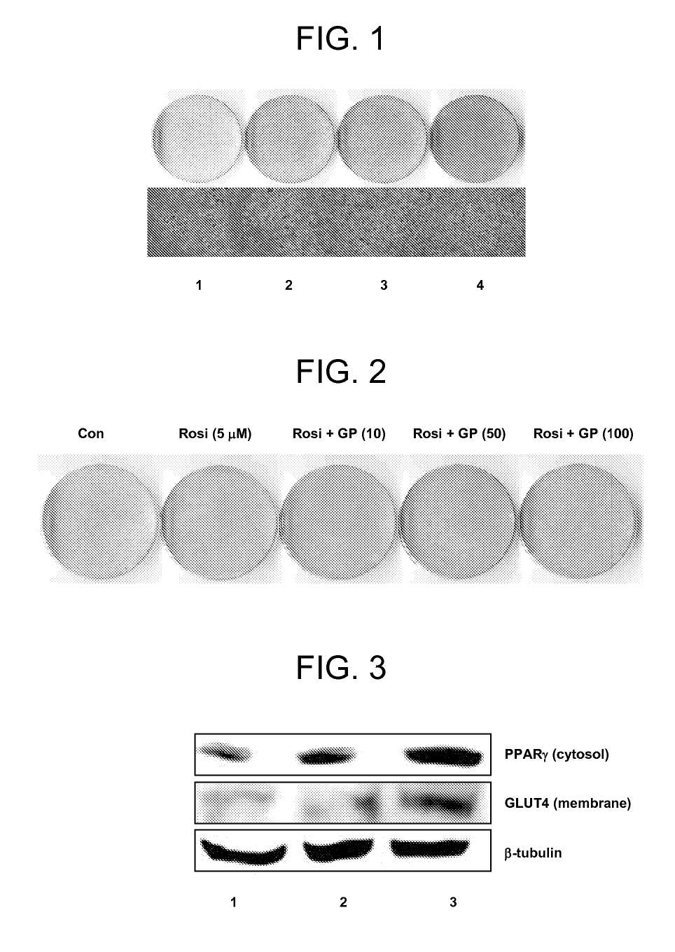

Adipogenecity of 3T3-L1 cells increased proportionally as the added amount of GP increased (FIG. 1). This data implicates that GP has an ability to stimulate glucose uptake into cells to meet carbon source requirement for triglycerides synthesis in the cells, since the sole energy source required for the accumulation of triglycerides inside cells is glucose in in vitro cell culture. When the cells were triggered by GP in the presence of rosiglitazone, the adipogenesis increased further (FIG. 2). This result suggests that GP differs with rosiglitazone in triggering mechanism of adipogenesis. We supposed that materials that can increase adipogenesis may increase glucose uptake in the major glucose disposing tissues to meet the physiological demand. Adipogenesis in 3T3-L1 cells is an active cellular differentiation process, implying the materials enhancing adipogenesis may not be toxic to cellular physiology. Not surprisingly, many adipogenesis stimulating materials also boosted GLUT4 translocation.

Circulating glucose after meal is taken up by muscle and other insulin sensitive tissues. Rat vascular smooth muscle cells, which are insulin-sensitive cells, were prepared to measure whether the candidate compounds can increase GLUT4 translocation to plasma membrane in two ways. First, membranous fraction lacking microsomes were prepared by differential centrifugation (Pinent, 2004). Equal amount of membrane protein was loaded on each lane of SDS-PAGE and transferred to nitrocellulose membrane. The blotted membrane was then reacted with anti-GLUT4 antibody and the specific bands were visualized on a film exposed to fluorescence radiated by HRP-conjugated secondary antibodies with appropriate substrates. In this experiment, G. pentaphyllum extract treatment increased membranous GLUT4 conclusively compared with control cells (FIG. 3 middle panel), indicating that the GP treatment stimulated GLUT4 translocation. As stated in our earlier experiment, G. pentaphyllum extract also up-regulated the PPARγ level, which is an important factor for enhancing adipogenesis (FIG. 3 upper panel).

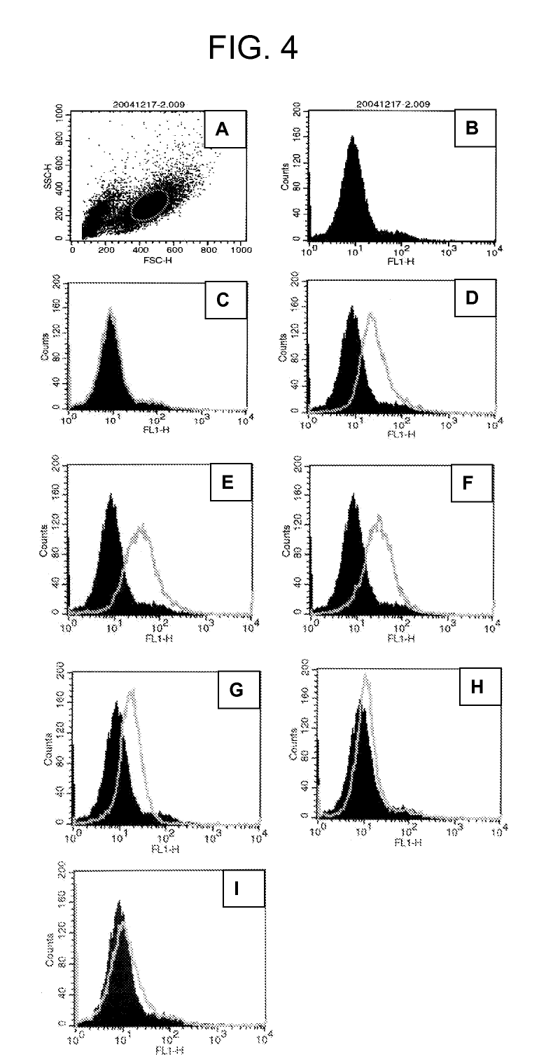

In an alternative way, L6 myotube cells were reacted with anti-GLUT4 antibodies and subsequently decorated with FITC-conjugated anti-rabbit IgG secondary antibodies. The fluorescence intensity was determined in FACS analysis. Insulin was used as a positive marker for GLUT4 translocation. GP treatment boosted GLUT4 translocation (FIG. 4C & D). Interestingly, GLUT4 translocation enhanced by GP treatment was not inhibited by either wortmanin (FIG. 4E), an inhibitor of PI3K, or SB20358, which is an inhibitor of p38 MAPK activity (FIG. 4F). Since PI3K and p38 MAPK are important mediators of insulin signaling, the pathway of GLUT4 translocation by GP is probably different from that of insulin (FIG. 4G, H, I).

### Example 3

### GP Alleviates Insulin Resistance Induced by Either High Glucose or Free Fatty Acid in Vitro

AMPK directly modulates ACC and 3-hydroxy-3-methyl-glutaryl-CoA reductase (HMG-COA reductase) by phosphorylation (Henin, 1995), resulting in increase of β-oxidation in mitochondria and reduction of cholesterol synthesis in hepatocytes, respectively. An AMP analog, 5′-phosphoribosyl-5-aminoimidazole-4-carboxamide (AICAR) has been found to stimulate AMPK.

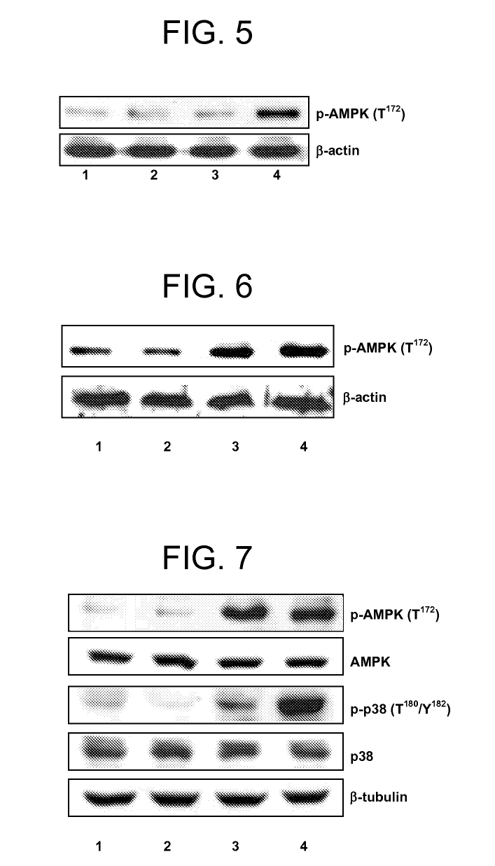

In a previous example, GP triggered GLUT4 translocation to plasma membrane. Besides insulin action, muscle contraction has been known to stimulate GLUT4 translocation via AMPK activation. We treated GP on L6 myotube cells to determine whether the treatment activates AMPK activity. Activation of AMPK was estimated by the increase in the phosphorylation on the threonine¹⁷² residue of AMPK using specific antibodies. AMPK phosphorylation was exhibited within 2 hs of GP treatment in L6 myotube cells (FIG. 5) and its level of phosphorylation was not increased with longer treatment of GP. Cells were pretreated with high glucose (27.5 mM) to induce insulin resistance and to repress AMPK activation (Itani, 2003). There was a marked increase in AMPK phosphorylation at the threonine residue upon treatment with GP. Higher dose of GP treatment induced more phosphorylation of AMPK. These data indicate that GP clearly activates AMPK activity in insulin sensitive cells (FIG. 5 and 6).

L6 cells were differentiated into mature muscle cells in a low serum condition (2%, v/v). AICAR was used as an internal positive control. Once drug treatments were completed, cells were harvested, subjected to be lysated, and equal amount of proteins from each treatment was loaded on gels for analyzing AMPK and p38 MAPK activity levels (FIG. 7). Total AMPK protein was also immunoblotted to make sure that equal amount of protein was loaded on the gels. At 30 μg/mL of GP L6 cells did not show an increase in phosphorylation on the threonine¹⁷² of AMPK compared with no treatment. However, at 60 μg/mL of GP, the phosphorylation level on AMPK was increased significantly. Phosphorylation level of AMPK by AICAR at 1 mM was comparable to that of 60 μg/mL of GP. GP is a mixture of similar compounds sharing a common backbone structure. GP appears to show a higher potency than AICAR in activating AMPK assuming that the average M.W of GP is about 1,000 Dalton.

AICAR was reported to activate p38 MAPK in skeletal muscle tissue (Lemieux, 2003). It was demonstrated that p38 activation is involved in the enhanced glucose uptake by AICAR. We investigated whether GP is able to activate p38 MAPK in L6 cells. Treatment with AICAR indeed exhibited p38 activation, while GP barely increased p38 activation (FIG. 7). In a previous example (FIG. 4), we have shown that SB20358, an inhibitor of p38 MAPK activity, did not block the GLUT4 translocation enhancement by GP. Considering the differential responses between GP and AICAR on AMPK (GP>AICAR) and on p38 MAPK (GP<AICAR), the molecular mechanism of GP on stimulating GLUT4 translocation of muscle cells may not be identical to that of AICAR.

### Example 4

### GP Effect on ACC

ACC is an important enzyme regulating lipid metabolism in various tissues, especially in the liver and muscles. The enzyme carboxylates acetyl-CoA to produce malonyl-CoA, which inhibits CPT-1 in outer mitochondrial membrane. The CPT-1 activity is known to be the rate-limiting step for fatty acids oxidation in the mitochondria (Lehninger, 2000). ACC is a target protein for AMPK kinase activity (Fryer, 2002). When muscle contracts, muscle cells trigger AMPK stimulation (Vavvas, 1997) to infuse more ATP through fatty acid combustion. Inactivation of ACC by AMPK is a key target point for reducing fatty acids.

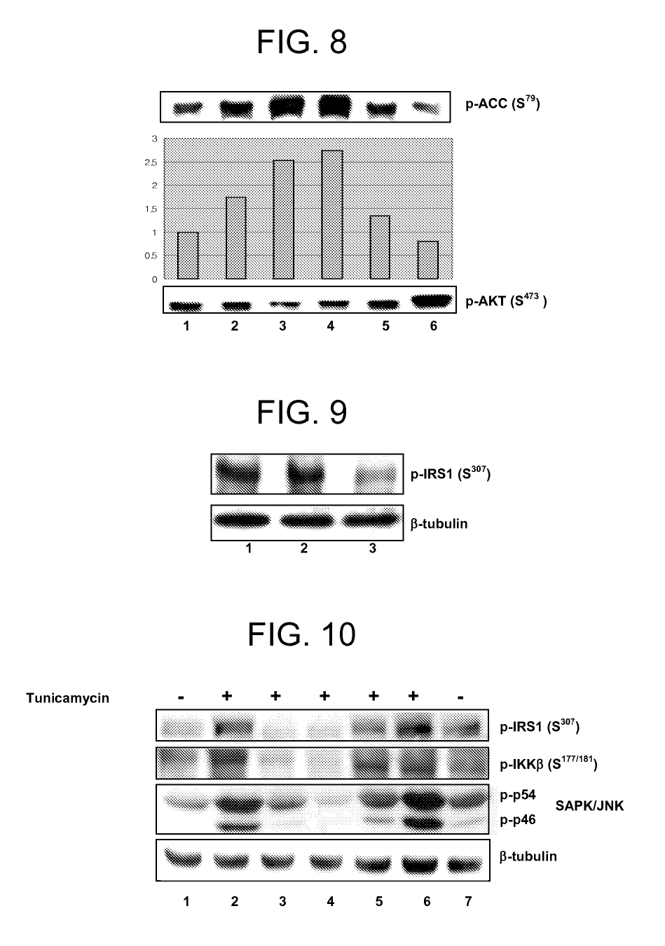

L6 myotube cells were treated with GP and AICAR. GP treatment clearly phosphorylated Ser⁷⁹ residue of ACC (FIG. 8). ACC phosphorylation peaked at 60 μg/mL of GP. AICAR treatment also increased the phosphorylation of ACC but slightly less than that observed with 30 μg/mL of GP. Insulin did not affect the phosphorylation. This data indicates that GP treatment on muscle cells mimics muscle contractions at molecular level.

AKT (also called protein kinase B, PKB) phosphorylation is catalyzed by PI3K in insulin signal pathway. Whether GP treatment on the muscle cells affects AKT activity was tested. Neither GP nor AICAR appeared to affect AKT activity (FIG. 8). Insulin, a positive control, activated AKT activity considerably. This experiment demonstrates that GLUT4 translocation (as illustrated in FIGS. 3 & 4) stimulated by GP seems to be not related to the insulin signaling pathway, rather the event mimics muscle contraction.

### Example 5

### GP Reduces Insulin Resistance in Muscle Cells by Repressing IKK Activity

Insulin resistance is a crucial metabolic abnormality in most metabolic syndrome including type 2 diabetes and hypertension. Evidence is mounting that attenuating the risk of insulin resistance reduces cardiovascular disorders (Reaven, 2005). Therefore, reducing insulin resistance, mostly manifested in insulin responsive tissues, such as skeletal muscles, liver, and adipocytes, may improve health conditions. The molecular mechanisms underlying developing insulin resistance among insulin responsive tissues are known to be different. Nonetheless, the final molecular markers are the same, phosphorylation of serine residues on IRS1. Two important transducers of insulin resistance in muscle cells are kinases; IKKβ and JNK (Gual, 2005).

A typical marker for insulin resistance manifests serine phosphorylation on IRS1 in muscle cells. There was a slight increase in the Ser³⁰⁷ phosphorylation on IRS1 of the cells treated with fatty acid conjugated BSA compared with those with BSA alone. Treatment of GP (60 μg/mL) on the cells markedly decreased the Ser³⁰⁷ phosphorylation level of IRS1 (FIG. 9). The reduction of Ser³⁰⁷ phosphorylation of IRS1 implies that the proximal insulin signal molecule, phosphatidylinositol 3-kinase, has a better chance of being recruited to the IRS1 (Pirola, 2003), of which event would render cells insulin sensitive. Although GP does not increase insulin sensitivity on muscle cells directly, the present evidence indicates that GP is capable of decreasing insulin resistance in muscle cells.

We investigated the molecular mechanism of the amelioration of insulin resistance by GP. There are two well-known kinases for serine-phosphorylation on IRS1, IKK complex and JNK (Gao, 2002). Recently it was described that JNK mediates obesity-derived impairment of insulin action in the liver and adipocytes (Ozcan, 2004). IKKβ was reported to phosphorylate not only IRS but also I-κB (Itani, 2002). Serine phosphorylation of IRS is related to insulin resistance directly and I-κB phosphorylation releases NF-κB, a key component in tissue inflammation. Some researchers consider type 2 diabetes as a chronic inflammatory disorder (Dandona, 2004; Sinha, 2004). We examined whether the reduced serine phosphorylation on IRS by GP is related to IKK activity, since GP is known to reduce inflammation induced by LPS in monocytes by repressing NF-κB activity (Aktan, 2003).

Tunicamycin, an antibiotic known to inhibit N-linked glycosylation, forces cells into an insulin-resistant status (Ozcan, 2004). The effect of GP on the phosphorylation of IKKβ in L6 myotube cells in the presence of tunicamycin was evaluated. The cytosolic fraction was subjected to SDS-PAGE and blotted on nitrocellulose membrane. The cytosolic fraction was subjected to SDS-PAGE and blotted on nitrocellulose membrane. The membrane was incubated with anti-phospho-IKKβ (S^{177/181}), anti-phospho IRS1 (S³⁰⁷), and anti-phospho SAPK/JNK (T¹⁸³) antibodies. Upon treatment of GP the Ser³⁰⁷ phosphorylation of IRS1 was dramatically reduced (FIG. 10, lanes 3 & 4 of upper panel). AICAR and insulin slightly reduced the serine phosphorylation of IKKβ. It was reported that Ser³⁰⁷ phosphorylation of IRS was mediated by IKKβ and/or JNK kinase activities in the muscle tissues of insulin resistant subjects. Therefore experiments were carried out to determine whether the reduced serine phosphorylation on IRS 1 by GP was associated with IKKβ and JNK activities. Not surprisingly, both kinase activities in the cells were significantly decreased by the treatment of GP. Further experiments were followed whether the GP's effect

Rat vascular smooth muscle cells were treated with GP in a high glucose medium, which is known to induce inflammation on vascular smooth muscle cells (Hattori, 2000). For measuring IKKβ activity, we investigated the level of phosphorylation on I-κB, a substrate of IKKβ. Nuclei-enriched fraction was obtained by a protocol as described elsewhere and the cytoplasmic fraction was obtained by a further centrifugation by removing microsomal membrane fraction. Equivalent amount of protein from each sample was loaded and resolved on a gel. The gel loaded with cytoplasmic fraction was immunoblotted for the specific phospho-Ser³² of I-κB and the nuclear fraction was immunoblotted for p65, a subunit of NF-κB.

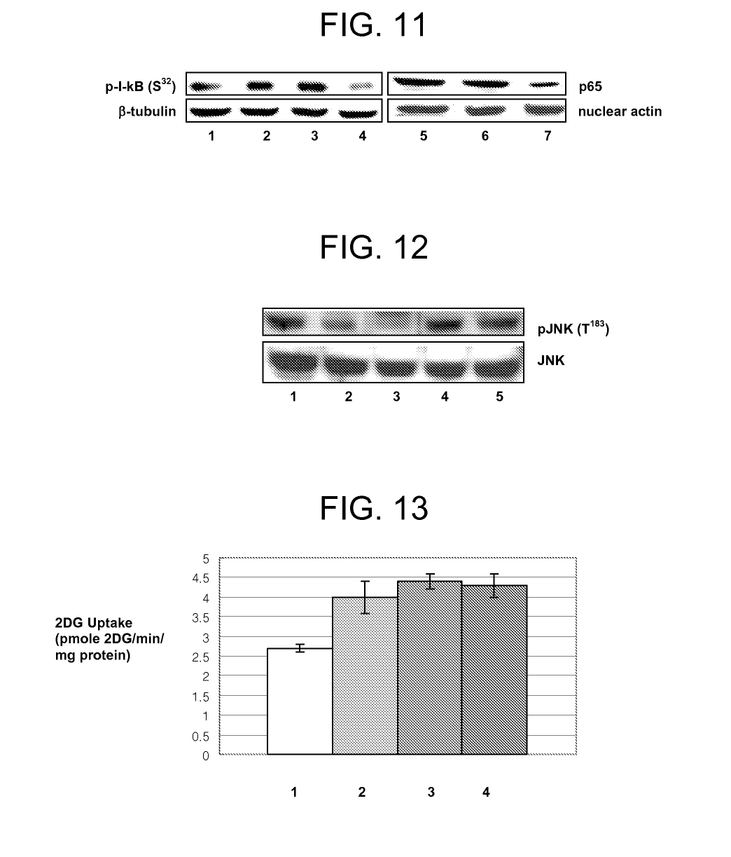

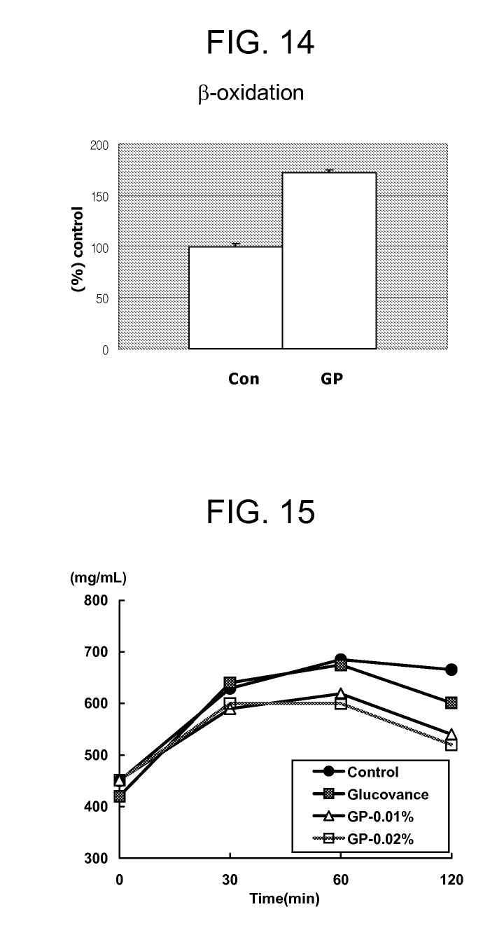

High glucose treatment slightly increased the phosphorylation on I-κB compared with that in normal concentration of glucose, implying that high glucose stimulated IKK activity. GP treatment at 10 μg/mL did not affect the phosphorylation on I-κB, but 30 μg/mL of GP significantly reduced the phosphorylation (FIG. 11, left panel), indicating reduced IKK activity. The nuclear fraction localized NF-κB was considerably reduced in the cells treated with 30 μg/mL of GP. GP treatment at 10 μg/mL did not affect NF-kB level in nuclei (FIG. 11, right panel). These observations are consistent with both the efficacy of the GP (I-κB phosphorylation level and NF-κB localization in nuclei) and the dose response (effective only at 30 μg/mL). These data address that GP reduces insulin resistance in muscle cells by repressing IKK activity.

We also investigated effects of GP treatment on JNK activity in L6 myotube cells. Intrinsic JNK activity was shown in L6 cells. There was substantial reduction of JNK activity in the cells treated with GP in dose-dependent manner (FIG. 12.). The JNK activity was not affected by either AICAR or insulin treatment. Taken together, GP reduced serine phosphorylation of IRS by inhibiting IKKβ and JNK activities.

### Example 6

### GP Increases Glucose Uptake by Stimulating AMPK

Examples 1-5 indicate that GP would increase glucose uptake regardless of the presence of insulin. To measure the glucose uptake in vitro, we performed 2-deoxyglucose uptake experiment. Since 2-deoxyglucose is not metabolized inside cells, radiolabeled 2-deoxyglucose was used for measuring glucose uptake experiment.

Whether GP increases glucose uptake in L6 myotube cells was investigated. Prior to adding the materials, the cells were incubated in the presence of high glucose, since high glucose is known to obstruct glucose uptake in muscle cells (Itani, 2003). GP (60 μg/mL), AICAR (1 mM) and insulin (100 nM) were incubated for 2 hs, 1 h and 20 min, respectively. Immediately after washing in Hepes buffered saline (HBS), the cells were incubated with 2-deoxyglucose (10 μM) in HBS for 10 min. Extensive washing was preceded before measuring radioactivity in scintillation counter. The uptake unit was estimated by the total counts of incubation.

High glucose did not affect the deoxyglucose uptake of L6 cells as has been reported by Itani et al. (FIG. 13). GP, AICAR and insulin treatments increased 2-deoxyglucose uptake by 24, 42, 40% on average, respectively. This experiment indicates that GP is capable of increasing glucose uptake in muscle cells probably by stimulating AMPK and/or p38 MAPK activities. This figure evidences that GP increased glucose uptake in muscle cells as much as insulin but via different mechanism. The concentration of treated GP was considerably lower than that of AICAR. In this regard the potency of GP on glucose uptake in muscle cell may be greater than that of AICAR.

In previous examples, GP activated AMPK and suppressed ACC activities, implying that GP provide an environment where β-oxidation increases. Whether GP treatment on hepatoma cell line HepG2 increase β-oxidation rate was assessed employing the method used previously (Singh, 1994). Cells treated with GP exhibited a marked increase by 70% of β-oxidation compared with cells without treatment. This data indicates that GP might reduce fat mass in an appropriate condition.

### Example 7

db/db mice, defect in functional leptin receptor, were used as a obese, hyperglycemic and insulin resistant animal model. The mice were fed with normal chow diet. GP and glucovance (a clinically approved medicine) as a reference drug were premixed with normal chow at the indicated ratios. The animals were fed ad libitum. Each cohort comprised 10 mice and bled once or twice to measure blood sugar concentrations during the adaptation period. The oral administration was continued for 8 weeks. At the time of sacrifice, Glucovance administered group showed weight loss by 22% on average, and GP administered groups also showed a 12% loss of weight compared with no treatment group (Table 1). There was no difference in food intake between groups. The cohort fed with GP showed reduced weight gain.

TABLE 1

| groups | Body weight(g) | Food intake(g/day) |
|---|---|---|
| Control | 38.05 ± 1.61^{a} | 4.29 ± 0.10^{NS} |
| Glucovance | 29.75 ± 1.09^{c} | 4.32 ± 0.11^{NS} |
| GP - 0.01% | 33.80 ± 0.80^{b} | 4.59 ± 0.13^{NS} |
| GP - 0.02% | 33.56 ± 1.14^{b} | 4.27 ± 0.10^{NS} |

^{abc}Values not sharing a common letter are significantly different among groups at p < 0.05
^{NS}Values are not significantly different groups at p < 0.05

Since a significant loss of body weight in groups administered with GP was noticed, we scored fat tissue amount whether the loss of body weight is attributable to a change in fat tissue amount. Table 2 shows that part of the weight loss of the animals may due to the decrease in epididymal and perirenal fat tissues amount. GP administered mice exhibited 1415% and 3538% decrease in weight of epididymal and perirenal fat tissues compared with animals with no treatment, respectively. The reduction in fat tissue weight may be related with the AMPK activation, resulting in an increase of β-oxidation.

TABLE 2

| | Epididymal fat | Perirenal fat |
|---|---|---|
| groups | (g/40 g B.W) | (g/40 g B.W) |
| Control | 2.00 ± 0.09^{a} | 1.05 ± 0.07^{a} |
| Glucovance | 1.75 ± 0.08^{b} | 0.72 ± 0.02^{b} |
| GP - 0.01% | 1.72 ± 0.06^{b} | 0.68 ± 0.03^{b} |
| GP - 0.02% | 1.70 ± 0.02^{b} | 0.65 ± 0.03^{b} |

^{ab}Values not sharing a common letter are significantly different among groups at p < 0.05

Blood was withdrawn every two weeks to measure glucose level. A day before sacrifice, the animals were fasted for 16 hr for the glucose tolerance test, by which glucose disposal rate was determined. Briefly, glucose (0.5 g/kg body weight) was intra-peritoneally injected in each animal, and blood glucose level was measured at the indicated time after injection.

Improvement in glucose tolerance with GP is illustrated (FIG. 15). An hour after glucose infusion, 9.7% and 11.8% improvement was shown for the mice fed with 0.01% and 0.02% GP, respectively in glucose disposal compared with the control group. The improvement was enhanced further at 2 hrs after the glucose infusion, where 19% and 22% improvement in the 0.01% and 0.02% GP fed mice, respectively was shown. Meanwhile, Glucovance administered group did not show any improvement in glucose disposal at lhr after the infusion, but there was significant improvement at 2 hrs after the infusion by 10% over the control group. This data illustrates that glucose disposal rate in db/db mice with GP was higher than that with glucovance.

### Example 8

### Various Illustrations of GP Effectiveness Against Markers of Insulin Resistance Syndrome

The benefits of administration of GP were further illustrated with improved insulin, C-peptide, glycated hemoglobin concentration (HbA1c), and leptin levels at the time of sacrifice.

### Example 8.1

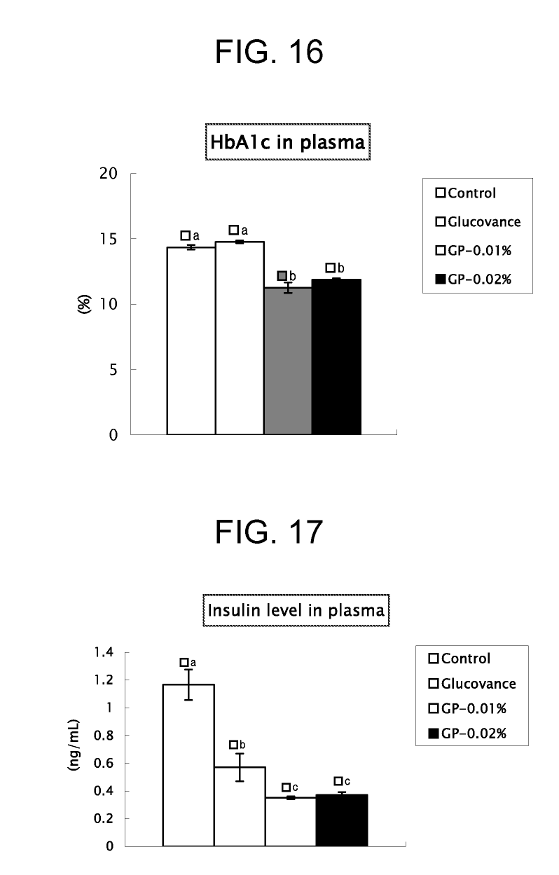

Glycated hemoglobin is a unique substance created as a result of interaction between hemoglobin and glucose. The hemoglobin A1C test is different from a fasting blood sugar test, which measures only the blood sugar level at the moment a sample is obtained. The AIC test, on the other hand, reflects average blood sugar level over longer periods. In a sense, the measurement of HbA1C decreases the risk of misinterpretation of diabetic status determined by the measurement of the blood glucose level. The glycated hemoglobin percentage was reduced by an average 17% and 16% in mice fed with 0.01% and 0.02% GP, respectively, compared with control group (FIG. 16). The reference drug, glucovance, surprisingly did not reduce the HbA1c level at all.

### Example 8.2

The tested animals, db/db mice, are congenitally malfunctioning in leptin signaling, therefore, the animals do not regulate their feeding behavior. Consequently, the animals become obese and show hyperlipidemia, hyperinsulinemia, and hyperleptinemia. Insulin resistance is an impaired metabolic response to a situation, where the blood insulin level is chronically higher. This disorder is associated very often with obesity, hypertension, abnormal triglycerides, glucose intolerance and type 2 diabetes. In this embodiment of the invention, GP alleviated the hyperinsulinemia associated with db/db mice (FIG. 17). In tested animals, GP treatment impressively reduced the blood insulin level by near 80% both in 0.01% and 0.02% GP fed group. Together with previous observation, GP improves insulin resistance convincingly.

### Example 8.3

When insulin is synthesized by the beta cells of the pancreas, it is produced as a large molecule (a propeptide). This molecule is then split into two pieces, insulin and C-peptide. The function of C-peptide is not known. The C-peptide level may be measured in a patient with type 2 diabetes or related disorders to see if any insulin is still being produced by the body. It may also be measured in the evaluation of hypoglycemia (low blood sugar) to see if the person's body is producing too much insulin. All groups of animals produced C-peptide. The reduced C-peptide level of the groups fed with GP supports the insulin lowering effect of GP (FIG. 18).

### Example 8.4

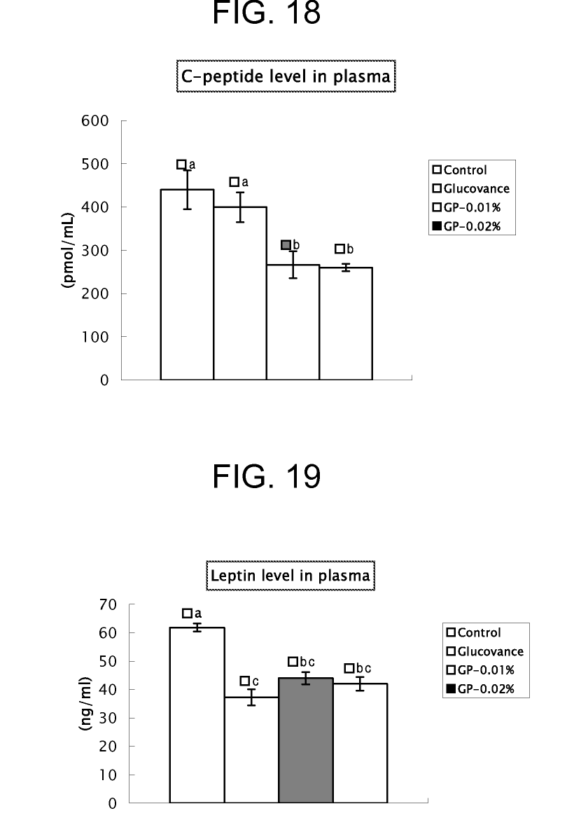

Leptin is an appetite-suppressing hormone secreted by adipocytes. However, most obese people are resistant to leptin rather than deficient in it. Resistance is associated with loss of function at several stages of the leptin-signaling pathway. Leptin's transport across the blood brain barrier is impaired by high triglycerides, and there is reduced function of the leptin receptor and its downstream targets (Banks, 2004). Insensitivity to leptin, which helps the body regulate its fat stores, contributes to obesity in mice. Leptin resistance could lead to other, more severe health conditions such as heart disease or diabetes. Leptin is comparatively highly expressed in ob/ob mice, which exhibit hyperinsulinemia (Mizuno, 2004). Taking this into consideration, db/db mice were GP treated with GP and leptin levels determined to see if GP treatment reduces both insulinemia and leptin levels. Leptin level of GP treated group was reduced significantly compared with those of animals with no treatment (FIG. 19), the mechanism of which is probably related to the reduced level of insulin.

### Example 9

### PEPCK Assay

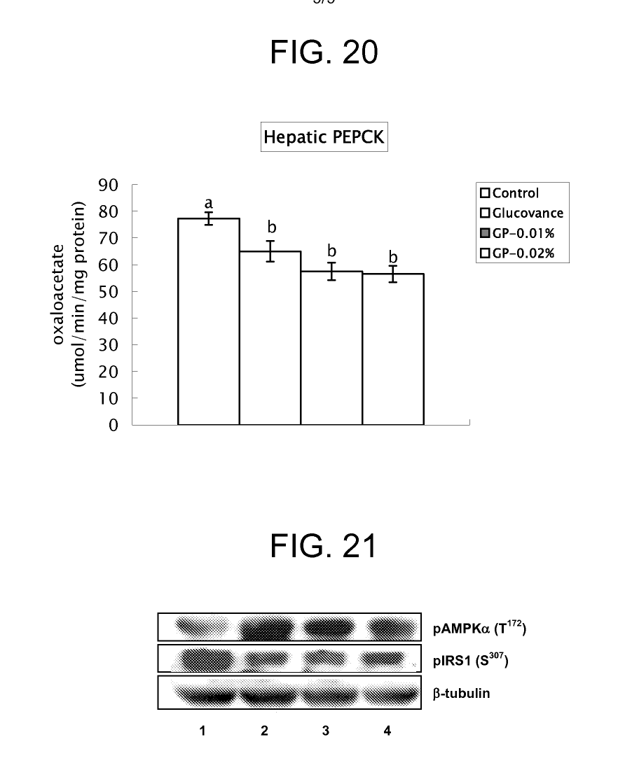

Hepatic phosphoenolpyruvate carboxykinase (PEPCK) is the rate-limiting step of gluconeogenesis. When PEPCK was overexpressed in liver, the tissue was less sensitive to insulin, indicating more glucose is produced in spite of higher insulin level (Sun, 2002). Lochhead and colleagues demonstrated that AICAR down-regulated PEPCK and glucose-6-phosphatase like insulin (Lochhead, 2000). PEPCK activity of the liver tissue was measured to determine whether GP affects its enzyme activity as has been observed with AICAR. When the production of oxaloacetate in the homogenized liver tissues in the presence of saturated phosphoenolpyruvate was measured, the GP treated liver showed 25% or less activity were scored compared with that of control animals (FIG. 20), demonstrating that GP may modulate gluconeogenesis via activating AMPK activity

### Example 10

### GP Administered Animals Showed Increased AMPK Activity and Reduced Serine Phosphorylation on IRS1.

Finally, AMPK activity in skeletal muscles of the tested animals was investigated. Simultaneously it was also determined whether the tissues of tested animals show reduced IRS serine phosphorylation as has been shown in cell experiments by GP treatment. Not surprisingly, the muscle tissues of GP administered animals revealed increased AMPK activity and lowered level of phospho-IRS, implying that administration of GP improved the insulin resistant state of the tested animals (FIG. 21).

### REFERENCES

Banks W A, Coon A B, Robinson S M, Moinuddin A, Shultz J M, Nakaoke R, Morley J E. Triglycerides induce leptin resistance at the blood-brain barrier. Diabetes. 2004, 53, 1253-1260.

Clarke P R, Hardie D G. Regulation of HMG-CoA reductase: identification of the site phosphorylated by the AMP-activated protein kinase in vitro and in intact rat liver. EMBO J. 1990. 9, 2439-2446.

Dandona P, Aljada A, Bandyopadhyay A. Inflammation: the link between insulin resistance, obesity and diabetes. Trends Immunol. 2004, 25, 4-7.

Dresner A, Laurent D, Marcucci M, Griffin M E, Dufour S, Cline G W, Slezak L A, Andersen D K, Hundal R S, Rothman D L, Petersen K F, Shulman G I. Effects of free fatty acids on glucose transport and IRS-1-associated phosphatidylinositol 3-kinase activity. J. Clin. Invest. 1999, 103, 253-259.

Fryer L G, Foufelle F, Barnes K, Baldwin S A, Woods A, Carling D. Characterization of the role of the AMP-activated protein kinase in the stimulation of glucose transport in skeletal muscle cells. Biochem. J. 2002, 363, 167-174.

Gao Z, Hwang D, Bataille F, Lefevre M, York D, Quon M J, Ye J. Serine phosphorylation of insulin receptor substrate 1 (IRS-1) by inhibitor KappaB kinase (IKK) complex J. Biol. Chem., 2002, 277, 48115-48121.

Goetze S, Kintscher U, Kaneshiro K, Meehan W P, Collins A, Fleck E, Hsueh W A, Law R E. TNFalpha induces expression of transcription factors c-fos, Egr-1, and Ets-1 in vascular lesions through extracellular signal-regulated kinases 1/2. Atherosclerosis. 2001, 159, 93-101.

Gual P, Le Marchand-Brustel Y, Tanti J F. Positive and negative regulation of insulin signaling through IRS-1 phosphorylation. Biochimie. 2005, 87, 99-109.

Hardie D G. Regulation of fatty acid synthesis via phosphorylation of acetyl-CoA carboxylase. Prog Lipid Res. 1989, 28, 117-146.

Hattori Y, Hattori S, Sato N, Kasai K. High-glucose-induced nuclear factor kB activation in vascular smooth muscle cells. Cardiovasc. Res. 2000, 46, 188-197.

Henin N, Vincent M F, Gruber H E, Van den Berghe G. Inhibition of fatty acid and cholesterol synthesis by stimulation of AMP-activated protein kinase. FASEB J. 1995, 9, 541-546.

Hotamisligil G S. Mechanisms of TNF-alpha-induced insulin resistance. Exp Clin Endocrinol Diabetes. 1999, 107, 119-125.

Itani S I, Saha A K, Kurowski T G, Coffin H R, Tornheim K, Ruderman N B. Glucose autoregulates its uptake in skeletal muscle: involvement of AMP-activated protein kinase. Diabetes 2003, 52, 1635-1640.

Kyriakis, J M., At the crossroads: AMP-activated kinase and the LKB1 tumor suppressor link cell proliferation to metabolic regulation. J. Biol. 2003, 2, 26.

Lehninger, Principles of Biochemistry, 2000, 3rd Ed., pp 599-605, Worth Publishers, NY.

Lemieux K, Konrad D, Klip A, Marette A. The AMP-activated protein kinase activator AICAR does not induce GLUT4 translocation to transverse tubules but stimulates glucose uptake and p38 mitogen-activated protein kinases alpha and beta in skeletal muscle. FASEB J. 2003, 17, 1658-1665.

Liu X., Ye W, Mo Z, Yu B, Zhao S, Wu H, Che C, Jiang R, Mak T C and Hsiao W L. Five new ocotillone-type saponins from Gynostemma pentaphyllum J. Natural Products 2004, 67, 1147-1151.

Lochhead P A, Salt I P, Walker K S, Hardie D G, Sutherland C. 5-aminoimidazole-4-carboxamide riboside mimics the effects of insulin on the expression of the 2 key gluconeogenic genes PEPCK and glucose-6-phosphatase. Diabetes. 2000, 49, 896-903.

Kim J K, Kim Y J, Fillmore J J, Chen Y, Moore I, Lee J, Yuan M, Li Z W, Karin M, Perret P, Shoelson S E, Shulman G I. Prevention of fat-induced insulin resistance by salicylate. J. Clin. Invest. 2001, 108, 437-446.

McGarry J D. Banting lecture 2001: dysregulation of fatty acid metabolism in the etiology of type 2 diabetes.Diabetes. 2002, 51, 7-18.

Mizuno T M, Funabashi T, Kleopoulos S P, Mobbs C V. Specific preservation of biosynthetic responses to insulin in adipose tissue may contribute to hyperleptinemia in insulin-resistant obese mice. J. Nutr. 2004, 134, 1045-1050.

Oh W, Abu-Elheiga L, Kordari P, Gu Z, Shaikenov T, Chirala S S, Wakil S J. Glucose and fat metabolism in adipose tissue of acetyl-CoA carboxylase 2 knockout mice. Proc Natl Acad Sci U.S.A. 2005, 102, 1384-1389.

Ozcan U, Cao Q, Yilmaz E, Lee A H, Twakoshi N N, Ozdelen E, Tuncman G, Gorgun C, Glimcher L H, Hotamisligil G S. Endoplasmic reticulum stress links obesity, insulin action, and type 2 diabetes. Science. 2004, 306, 457-461.

Pirola L, Bonnafous S, Johnston A M, Chaussade C, Portis F, Van Obberghen E. Phosphoinositide 3-kinase-mediated reduction of insulin receptor substrate-1/2 protein expression via different mechanisms contributes to the insulin-induced desensitization of its signaling pathways in L6 muscle cells. J. Biol. Chem. 2003, 278, 15641-15651.

Reaven G M. The insulin resistance syndrome: Definition and dietary approaches to treatment. Ann. Rev. Nutrition 2005, 25, 391-406.

Singh H, Beckman K, Poulos A. Peroxisomal beta-oxidation of branched chain fatty acids in rat liver. J. Biol. Chem. 1994, 269, 9514-9520.

Sinha S, Perdomo G, Brown N F, O'Doherty R M. Fatty acid-induced insulin resistance in L6 myotubes is prevented by inhibition of activation and nuclear localization of nuclear factor kappa B. J. Biol. Chem. 2004, 279, 41294-41301.

Sun Y, Liu S, Ferguson S, Wang L, Klepcyk P, Yun J S, Friedman J E. Phosphoenolpyruvate carboxykinase overexpression selectively attenuates insulin signaling and hepatic insulin sensitivity in transgenic mice. J. Biol. Chem. 2002, 277, 23301-23307.

Vavvas D, Apazidis A, Saha A K, Gamble J, Patel A, Kemp B E, Witters L A, Ruderman N B. Contraction-induced changes in acetyl-CoA carboxylase and 5′-AMP-activated kinase in skeletal muscle. J. Biol. Chem. 1997, 272, 13255-13261.

Weisberg S P, McCann D, Desai M, Rosenbaum M, Leibel R L, Ferrante A W Jr. Obesity is associated with macrophage accumulation in adipose tissue. J. Clin. Invest. 2003, 112, 1796-1808.

Yin F, Hu L, Lou F, Pan R. Dammarane-type glycosides from Gynostemma pentaphyllum. J. Nat. Prod. 2004, 67, 942-952.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A composition comprising an insulin resistance syndrome, obesity, decreasing body fat mass and hypertriglyceridemia treating effective amount of an extract of Gynostemma pentaphyllum.

2. The composition according to claim 1, wherein the composition comprises gypenosides in a concentration of about 0.5 to 10% by weight.

3. The composition according to claim 1, wherein the amount of gypenosides in the composition is about 10 to 2,000 μg/ml μg/ml.

4. A method for treating symptoms of insulin resistance syndrome, obesity/overweight and hypertriglyceridemia in a subject administering to the subject a therapeutically effective amount of the composition according to claim 1.

5. The method according to claim 4, wherein the amount of the extract is 10 mg to 30 g per day.

6. The method according to claim 5, wherein the amount of the extract is about 0.5 g to 5 g per day.

7. A method for treating symptoms of insulin resistance syndrome, obesity/overweight, decreasing body fat mass and hypertriglyceridemia in a subject comprising administering to the subject a therapeutically effective amount of gypenosides composition from the composition according to claim 1.

8. The method according to claim 7, wherein the amount of the gypenosides composition is about 1 to 1000 mg per day.

9. The method according to claim 8, wherein the amount of the gypenosides composition is 10 to 800 mg per day.

10. The composition according to claim 1, comprising an aqueous carrier selected from the group consisting of spring water, filtered water, distilled water, carbonated water, juice, yogurt, milk, edible oils and a combination thereof.

11. The composition according to claim 1, comprising as food additives, ice cream, hamburger, cereals, cookies, breads, cakes, biscuits, meat product, or a combination thereof.

12. The composition according to claim 1, comprising a preservative agent, sweetener, flavoring agent, coloring agent, or a combination thereof.

13. The composition according to claim 1 formulated into a tablet.

14. The composition according to claim 13, wherein the tablet is made from a base selected from a group consisting of a filler, binder, coating, excipients, and a combination thereof.

15. The composition according to claim 14, wherein a base for the tablet is selected from the group consisting of plant cellulose, natural silica, magnesium sterate, wax, vegetable glycerides, vegetable stearate and a combination thereof.

16. The composition according to claim 1, comprising a compound selected from the group consisting of glitazones, fibrates, statins, biguanides, sulfonylureas, adenine nucleotides, their derivatives, and pharmaceutically acceptable salts thereof.

17. A method for selecting non-toxic AMPK activators, which have adipogenesis enhancing activity in 3T3-L1 cells.
